(19) United States
(12) Patent Application Publication
(10) Pub. No.: US 2007/0150032 A1
(43) Pub. Date: Jun. 28, 2007

(54) Laser with intelligent therapeutic fiber

(75) Inventors: Werner Hiereth, Gilching/Geisenbrunn (DE); Jurgen Austen, Freising (DE)

Correspondence Address: KING & SPALDING LLP, 1180 PEACHTREE STREET, ATLANTA, GA, 30309-3521, US

(73) Assignee: Dornier MedTech Laser GmbH, Wessling (DE)

(21) Appl. No.: 11/709,574

(22) Filed: Feb. 22, 2007

Related U.S. Application Data
(63) Continuation of application No. 10/673,913, filed on Sep. 29, 2003.

(30) Foreign Application Priority Data
Sep. 27, 2002 (DE) 102 45 140.0

(51) Int. Cl. A61N 5/06; A61B 18/18

(52) U.S. Cl. 607/89; 606/15

(57) ABSTRACT

A laser system is provided that includes a laser device for the generation of laser radiation and a light guide for guiding the generated laser radiation. A data medium for identity data is connected to the light guide, and a readout device is included for reading out the identity data. A light guide system is also provided that includes a light guide that can be releasably coupled to a laser device using a mounting device and a data medium for identity data connected to the light guide.

### RELATED APPLICATIONS

This application claims priority to co-pending German Patent Application No. 102 45 140.0, filed Sep. 27, 2002, and U.S. patent application Ser. No. 10/673,913, filed Sep. 29, 2003. The complete disclosure of each of the above priority applications is hereby fully incorporated herein by reference.

### FIELD OF THE INVENTION

The present invention generally relates to a laser system for medical applications, and more particularly, to a laser system with intelligent therapeutic fiber.

### BACKGROUND OF THE INVENTION

The ever increasing number of fields of application for laser technology in medicine are leading to the development of technically increasingly refined laser designs and corresponding system concepts which simplify and improve dealing with laser systems or which open up new fields of application. In this connection the application of flexible, optical transmission systems for the generated laser radiation takes on significant importance, because for applications of laser radiation at or in the place of therapy the distance between the laser device output and the patient must be bridged. Medical laser systems therefore typically consist of a stationary or mobile laser device, a beam guidance system, optical end devices, and accessories for special medical applications. For the transmission of visible laser light and the bordering spectral ranges from approx. 0.3 μm to 2.1 μm, flexible glass or quartz fibers are typically used. In the spectral ranges of 0.19 μm to 0.3 μm (excimer lasers) and 3 μm to 10 μm (erbium and CO₂ lasers) special light guides or mirrors mounted on articulated arms are typically used.

Particularly high requirements are usually placed on light guides in the transmission of pulsed, high-energy laser radiation. Ease of handling and versatility of these transmission systems is typically of crucial importance for the application of the laser systems. The light guides used here usually have the most varied specifications with regard to transmission properties, the maximum laser power that can be applied, the end date for usage of sterile fibers, etc. These specifications prescribe certain boundary conditions in relation to the applicability of certain types of light guides in combination with certain lasers or treatment parameters. Conformance to these boundary conditions is usually communicated to the user via the instructions for use supplied with the laser or light guide. The responsibility therefore usually resides with the user of a laser device and cannot be checked by a control system in the laser device.

The consequences for not allowing for or misinterpreting these boundary conditions by the user are, for example, damage to the fibers, too little laser power at the end of the fiber, or treatment with unsterilized fibers. The corresponding result may be unsuccessful treatment or direct impairment of a patient's health. If liability claims are then made by the user due to a malfunction of a damaged fiber or by a patient due to impairment of his or her health, differentiation may no longer be made retrospectively between a quality defect in the fiber and non-conformance to the boundary conditions for the application of the light guide by the user.

In this respect, expendable light guides for contact and contact-free laser therapy take on special importance, because they can be used without problem with endoscopes and other laser guidance instruments and due to their advantages are very popular. They can be used immediately, are packed in sterile packages and are supplied from the factory with traceable quality.

### SUMMARY OF THE INVENTION

It is therefore an object of this invention to provide a laser system and a light guide which simplify conformance to the boundary conditions for the use of a light guide in the laser system and to render erroneous operation of the laser system in conjunction with the light guide traceable or not possible. Exemplary embodiments of the invention are based on the concept that a transponder permanently connected to the light guide can transmit identification data to the laser device and the laser device can transmit parameter settings to the transponder of the light guide which can be evaluated at a later point in time.

The solution according to the invention can provide for a laser device to output a warning signal or carry out the laser device settings automatically based on the data transmitted from the transponder when there are incorrect settings of the boundary conditions in relation to the light guide. During the assessment of whether a quality defect in the light guide or an application error is involved, the parameter settings recorded when the light guide was used can be included. This can be particularly important for expendable light guides, because their quality and stressing limits are specified according to a therapeutic application.

According to one aspect of exemplary embodiments of the invention, a laser system is provided that includes a laser device for the generation of laser radiation and a light guide for guiding the generated laser radiation. A data medium for identity data is connected to the light guide. In addition, a readout unit for reading out the identity data is arranged in the laser device.

According to another aspect of exemplary embodiments of the invention, a light guide system is provided that includes a light guide for guiding laser radiation and a data medium for identity data permanently connected to the light guide. The light guide can be releasably coupled to a laser device using a mounting device

These and other aspects of the invention will be described further in the detailed description below in connection with the drawings and the claims.

### BRIEF DESCRIPTION OF THE DRAWINGS

The accompanying drawings are incorporated into and form a part of the specification for the purpose of explaining the principles of the invention. The drawings are not to be construed as limiting the invention to only the illustrated and described examples of how the invention can be made and used. Further features and advantages will become apparent from the following, and more particular description of the invention as illustrated in the accompanying drawings, wherein:

FIG. 1 shows a schematic illustration of a laser system according to exemplary embodiments of the invention.

FIGS. 2a and 2b show another schematic illustration of exemplary embodiments of the invention.

FIG. 3 shows a flow chart schematically representing a sequence of data communication according to exemplary embodiments of the invention.

FIG. 4 shows a schematic overview of exemplary application data saved in the transponder according to exemplary embodiments of the invention.

### DETAILED DESCRIPTION OF THE INVENTION

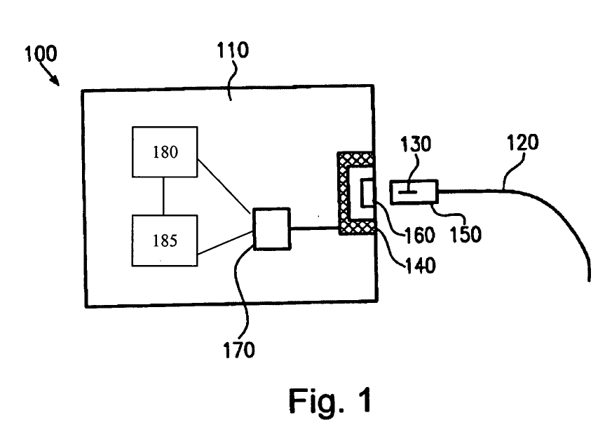

The illustrative embodiments of the present invention will be described with reference to the figure drawings, wherein like elements and structures are indicated with like reference numbers. FIG. 1 shows a schematic illustration of a laser system 100 according to exemplary embodiments of the invention and based on a schematic illustration. The laser system 100 includes a stationary or mobile laser device 110, which contains a device for the generation of laser radiation, to which a flexible laser guide 120 can be coupled for guiding a beam of generated laser radiation.

The laser device 110 can be equipped with high energy laser diodes, a micro-optical system for focusing the generated laser light, and a power supply for the generation of intensive laser radiation. Alternatively, the laser device 110 can be equipped with a laser medium, a resonator, a pump source, and the appropriate power supply. Diode-pumped solid-state laser media are preferable in this application for the generation of the intensive laser radiation.

The laser device 110 preferably also includes a cooling device and a system controller, the tasks of which include the control of the power of the laser radiation, the pulse duration, and the frequency of the laser pulse. Furthermore display and control devices can be integrated into the laser device 110, enabling the specific application modes and the system settings to be selected. In addition, the laser device 110 can include appropriate safety devices, both for the electrical and the optical sections. Preferably, the system controller possesses appropriate devices to enable the open and closed-loop control of the laser system 100 to be carried out by software programs. In this respect, some exemplary embodiments are particularly advantageous in which software programs can be replaced during an update.

In alternative exemplary embodiments of the invention, an output unit for a log of the system settings can be integrated into the laser device 110 or the laser device 110 that comprises an interface for an output unit. In addition, a mounting device 140, which can be used for permanent or releasable mounting of the light guide 120, can be integrated into the laser device 110. The mounting device 140 is preferably a plug, screw, or bayonet connection, whereby the part of the mounting device 140 mounted on the laser device 110 is preferably arranged as a socket 160 and the part mounted on the light guide 120 is formed as a plug 150. A so-called SMA connector can be used preferably for the releasable mounting of the light guide 120.

The light guide 120 can comprise one or more plastic, glass, or quartz-glass fibers. Depending on the wavelength of the generated laser radiation, doped quartz-glass fibers can be used. The light guide 120 is designed to be able to transport high luminous powers as loss-free as possible. For safety reasons, the light guide 120 can include a suitable sheath to protect the fibers from undue mechanical stress and to guard against the emission of laser radiation in the case of fiber breakage. The light guide 120 may preferably be a so-called expendable light guide 120, which is a therapeutic fiber packed in a sterile manner for use only once.

The plug 150 is preferably of a material which does not essentially screen electromagnetic radiation in the frequency range of a transmitter and receiver section of the transponder 130 and is preferably made, for example, of plastic. The plug 150 and the light guide 120 are typically connected together inseparably, and a transponder 130 can be accommodated in the plug 150. In this way, the glass fibers, the plug 150 of the mounting device 140, and the transponder 130 can be permanently connected together. Preferably, the transponder 130 can be permanently welded, glued, or encapsulated in the plug 150 of the mounting device 140 so that it cannot be removed.

The transponder 130 typically contains a read/write memory for recording all the relevant information which is generated during the manufacture of the therapeutic light guide 120 and during the application on the laser device 110. The laser device 110 is in this respect equipped with a circuit board for reading from and writing to the light-guide transponder 130. The data transmission occurs by wireless means, preferably in the RF 3.5 kHz band using an antenna. As mentioned above, data can be saved in the laser system 100 on an electronic data medium. Preferably, so-called radio-frequency identification (RFID) systems can be used in this regard. So-called transponder 130s can be fitted to the light guide 120 to be identified. The power supply for the transponder 130 and for the data interchange between the transponder 130 and the readout device 170 is typically not realized, however, through an electrically conductive contact but instead in a non-contacting manner using magnetic or electromagnetic fields.

The RFID system typically includes two components, which are the transponder 130 (mentioned above), which can be fitted to the light guide 120 to be identified, and a readout device 170 with antenna unit 140 which can be realized depending on the version both as a readout device and as a writing/readout device. This readout device 170 can alternatively be coupled to a local computer network. The readout device 170 can be preferably connected to the system controller of the laser device 110.

The readout device 170 preferably includes a control unit and a radio frequency (RF) interface. The principal task of the readout device 170 is the activation of the transponder 130, the establishment of a communication, and the transport of the data between the application software of the system controller for the laser device 110 and the contactless data medium. For both directions of data flow from and to the transponder 130, there are typically two separate signal trains within the RF interface available. Data that is transported to the transponder 130 can pass through the transmitter branch. In contrast, data that is received from the transponder 130 can be processed in the receiver branch.

In the RFID system, an interchange of data as well as energy can take place. Within the transponder 130, a converter is typically connected between the memory and the transmitter/receiver antenna, which converts the analog signals from the antenna into digital signals which can be used by the memory. The complete sequence can be monitored by control logic in a microchip in the transponder 130.

Outside of the response range of a readout device 170, the transponder 130 typically behaves passively, because it normally has no voltage supply of its own. It is usually only within the response range that is activated by the readout device 170 since the energy required for the operation of the transponder 130 is usually transmitted via a transmitter/receiver antenna. Preferably, the transponder 130 is programmable and without batteries (passive). Alternatively, transponder 130s with a fixed program, with or without batteries, or so-called semi-passive transponder 130s can be used in which the microchip is supplied from a battery, and for the data transmission, the electromagnetic field of the readout unit can be used inductively.

Depending on the application in the laser system 100, RFID systems are used with various ranges. For example, close-coupling systems with a very low range of up to approx. 0.01 m can be used. In this case, the transponder 130 is typically plugged into a readout device 170 or positioned on a surface provided for that purpose. Any frequencies up to 30 MHz can be used for the transmission. Due to the close coupling between the data medium and the readout device 170, large amounts of energy are typically made available for applications, which demand appropriate safety requirements, but do not need any long range.

Alternatively, remote coupling systems can be used that enable ranges of up to 1 m. These systems usually have the inductive coupling between the readout device 170 and the transponder 130 in common. Typically frequencies below 135 kHz and the region around 13.56 MHz are used as transmitter frequencies.

With another alternative embodiment, long range systems can be used in which ranges significantly more than 10 m are possible. In such case, the transmitted energy is typically not sufficient to supply the transponder 130 with sufficient energy for the operation of the microchip. Therefore, a back-up battery can provide energy exclusively for the microchip and the retention of the saved data (semi-active power supply). The transmitting frequencies here are typically in the microwave range (2.45-5.8 GHz).

As an economical alternative, a read-only transponder 130 can be preferably used. When the read-only transponder 130 is moved into the response range of the readout device 170, the output of a certain identification key (serial number) of the transponder 130 is initiated which was incorporated during the microchip production. Typically, this identification key and other data is written into the transponder 130 memory at the factory and cannot be changed.

As another alternative, a transponder 130 can be used that preferably can be written with data a number of times by the readout device 170 and is fitted with a read/write memory. The data transmission typically occurs in blocks. This means that a defined number of bytes are combined to form a block which then is read or written as a complete entity. This block structure enables a more simple addressing in the microchip and by the readout device 170. The memory size of the read/write transponder 130 varies depending on the application, and is typically between 1 byte and 64 kilobytes.

For applications of therapeutic fibers in which multiple rewriting is not necessary, a write-once transponder 130 can be alternatively used that can be written to once. To protect the saved data from undesired access, a so-called encryption unit, which can be used for identification, data encryption, and key management, can be preferably integrated into the microchip. Preferably, the encryption unit provides password protection and a 64-bit key set at the factory.

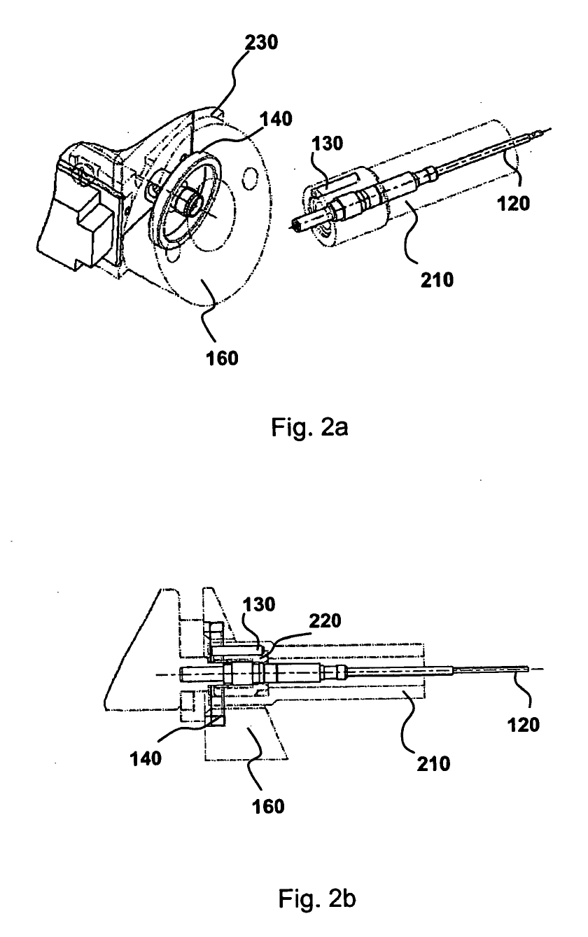

FIGS. 2a and 2b show another schematic illustration of exemplary embodiments of the invention. In these exemplary illustrations, the light guide 120 is permanently connected to the plug 150 and the plug housing 210. Preferably, the light guide 120 is connected to the plug 150 in an essentially non-releasable manner. The light guide 120 is in this case passed through the plug 150 and brought out at the open end of the plug 150 so that the generated laser radiation can be coupled to the light guide 120 at this end.

The transponder 130 is preferably encapsulated into the interior of the plug 150 with an encapsulation compound 220, so that it is connected to the light guide 120 and the plug 150 in an essentially inseparable manner. Alternatively, the transponder 130 can be welded into the plug housing 210 or glued to the plug housing 210. There are also other mounting possibilities that enable the transponder 130 to be connected to the light guide 120 and the plug 150 in a non-releasable manner or ensure that it is not possible to remove the transponder 130 from the plug 150 without damaging it. In this way it is ensured that the transponder 130 is coupled to the light guide 120 and the identification and application data saved in the transponder 130 is kept with the light guide 120. This makes it possible, for example, to prevent erroneous operation of the laser device 110 in conjunction with the light guide 120 and ensure that the history of the application of the light guide 120 can be traced back when needed.

In the laser device 110, the counterpart 160 for the plug connection 150 is fitted to the housing wall 230 of the laser device 110. As mentioned above, screw connections or other fastening devices can be alternatively used, and so-called SMA connectors can be preferably used here. Depending on the type of transponder 130 used, as mentioned above, a suitable transmitter and receiver device 140/170 can be arranged in the laser device 110. Preferably, an antenna 140 can be used which is fitted in the vicinity of the plug 150 or screw connection 150/160. In this way it can be ensured that the reception of the RFID system functions appropriately and reliably, and a sufficiently good signal-to-noise ratio is ensured. The transmitter and receiver device 140/170 and the transponder 130 can be arranged such that essentially they are not screened by the laser system 100 components, as depicted in FIG. 2b, so that an appropriately good reception in the RFID system can be ensured.

The antenna 140 can be coupled with a radio frequency interface, which in turn can be connected to a control unit. Reception and transmission data can be interchanged with the radio frequency interface by the control unit. The control unit can be preferably connected with the system controller of the laser device 110. It can then be possible for the light guide 120 data read out of the transponder 130 to be output via the radio frequency interface and passed to the system controller via the control unit. The system controller can indicate the necessary system settings by instructions on the display device or carry out appropriate system settings automatically, whereby erroneous operation of the laser device 110 with the light guide 120 used can be minimized.

Typically this is relevant to settings of the maximum pulse energy or duration and to the maximum number of laser pulses passed via the light guide 120 to the point of application. Furthermore, it can alternatively record whether the light guide 120 is a light guide 120 for multiple use or whether an expendable light guide 120 is being used. In the latter case, with the application of expendable therapeutic fibers, provision can alternatively be made for reading out and evaluating appropriate application data from the transponder 130 coupled to the expendable light guide 120. Moreover, for the case where the expendable therapeutic light guide 120 has been used, an appropriate warning signal can be displayed on the display device or the emission of a laser pulse via the light guide 120 can be inhibited.

In other alternative exemplary embodiments of the invention, the RFID system can be fitted to the end of the light guide 120 remote from the laser device 110, for example, when a light guide 120 is involved, to the end of which a plug/grip part combination for a so-called applicator can be fitted. In such case, the readout and writing of data can occur via an antenna and electronics unit accommodated in the grip part. Alternatively, the transmitter and receiver unit of the RFID system can also be directly accommodated in the laser device 110 if a remote coupling system with a range of up to 1 m or a so-called long range system with a greater range is used.

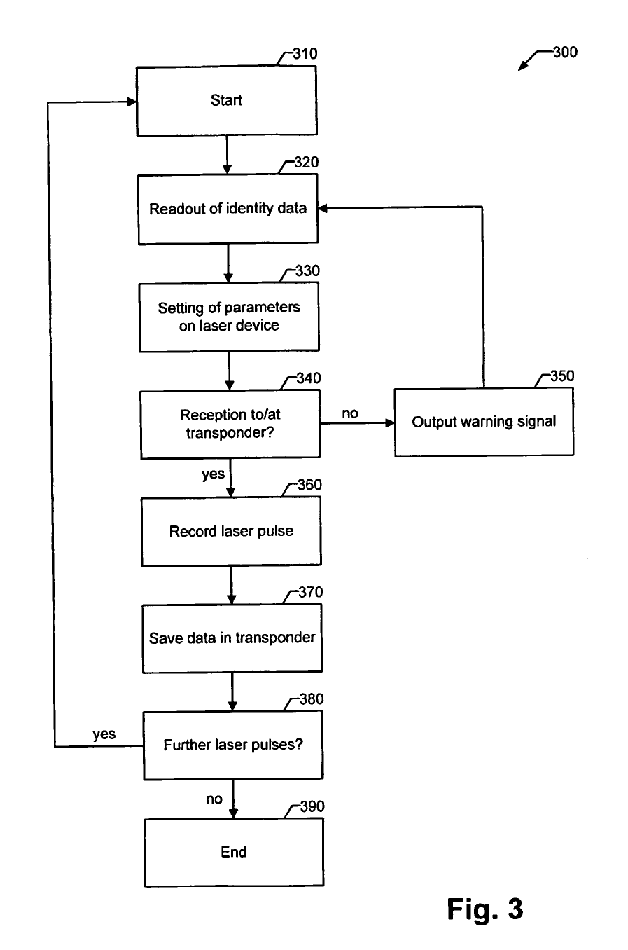

FIG. 3 shows a flow chart for the schematic sequence 300 of data communication between the transmitter and receiver device of the laser device 110 or of the above mentioned handpiece and the transponder 130 connected to the light guide 120 according to exemplary embodiments of the invention. In step 310 either the system controller of the laser device 110 or the control unit can initiate the start of the program routines. In step 320 the identity data can be read out of the transponder 130. If the readout of the identity data is not possible, an appropriate warning signal can be displayed on the display device or the emission of laser pulses can be inhibited.

Alternatively, in step 320 the application data can be additionally read out of the transponder 130. For the case in which an expendable light guide 120 is being used, a check can be made of whether appropriate application data has been saved in the transponder 130 or whether the expendable light guide 120 has already been used and appropriate data has been saved in the transponder 130. For this case an appropriate warning signal can be displayed on the display device or the emission of laser pulses can be inhibited. For the case in which a multiple-use light guide 120 is being used, the application data can be read out and a check can be made of whether the laser power emitted via the light guide 120 has exceeded a specified limit or the maximum number of applications for the guarantee of proper functioning of the light guide 120 has not yet been exceeded. For the case in which one of the figures is exceeded, as mentioned above, an appropriate warning signal can be displayed on the display device or the emission of laser pulses can be inhibited.

In step 330 the appropriate identity data can be passed via the control unit of the radio frequency interface to the system controller of the laser device 110. This identity data can preferably contain information about the manufacturer, the end date for usage, an average transmission power, a maximum transmission power, the type designation, and/or a fiber diameter of the light guide 120. Furthermore, additional data for the identification of the light guide 120, such as the production number, batch number, production date, or similar, can be saved in the transponder 130.

According to the data, the system controller can carry out, as already mentioned, system settings in the laser device 110, i.e. the laser power, pulse duration, or the maximum possible number of laser pulses can be automatically set. Alternatively, provision can be made in that with manual operation of the laser device 110, the system controller can output appropriate warning signals or correction suggestions via the display device when incorrect parameters are set. In this way it can be ensured that erroneous operation of the laser device 110 in conjunction with the light guide 120 is prevented. The risk of setting laser energies and laser pulse durations which would lead to the destruction of the light guide 120 or to an incorrect treatment is consequently minimized.

In step 340 the reception of the RF interface to/at the transponder 130 is checked. In this way it can be ensured that appropriate application data, such as for example, the laser pulse energy and laser pulse duration, can also be written into the transponder 130. If no reception to/at the transponder 130 is possible, an appropriate warning signal can be displayed via the display device in step 350. The sequence of the control then starts again at step 320 with the reading out of identification data from the transponder 130. If an appropriate reliable reception to/at the transponder 130 is established, the sequence continues with step 360.

In step 360, the appropriate system setting is recorded via the system controller and passed to the control unit of the radio frequency interface. In step 370, the application data determined by the system controller is passed to the transponder 130 and written to it. In step 380, the system controller or the controller of the radio frequency interface checks whether further laser pulses are emitted for the laser application. For the case where further laser pulses are emitted for the laser application, the controller continues with step 320. Otherwise the control process is terminated with step 390. Alternatively, an identification of the light guide 120 manufacturer can also be read out from the transponder 130 in step 320 and evaluated to check whether the light guide 120 was made by an authorized manufacturer.

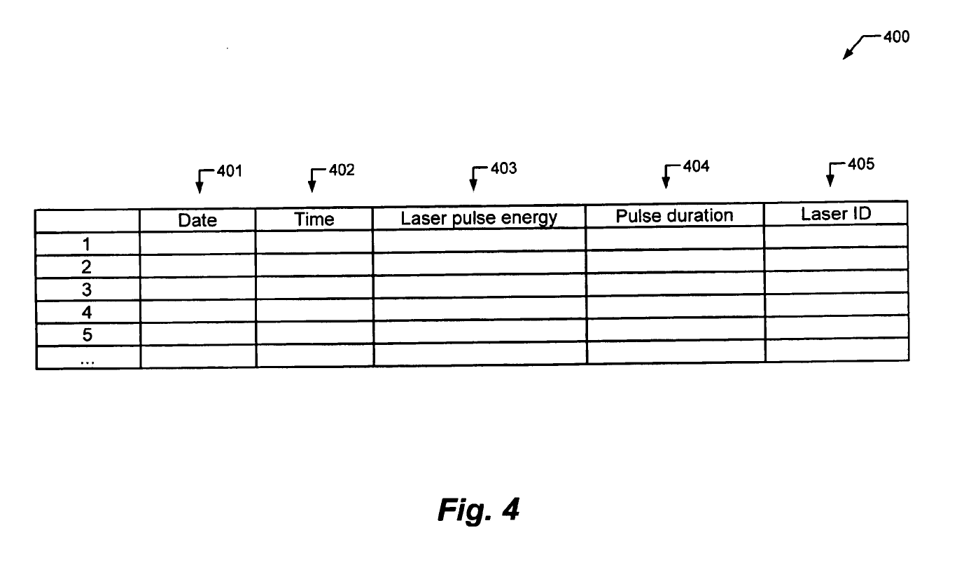

FIG. 4 shows a schematic illustration 400 of an overview of the application data saved in the transponder 130 according to exemplary embodiments of the invention. The system controller can determine the relevant date 401 and time 402 of the application as well as the corresponding laser pulse energy 403 and the laser pulse duration 404. This information can be transmitted together with an identification number 405 of the laser device 110 to the transponder 130 via the control unit of the RF interface. Here, each individual laser pulse, which has been emitted through the light guide 120 by the laser device 110, can be recorded in the transponder 130, as already described above and provided with an incremental number.

The saved data facilitates tracing the history of the light guide 120 application. To this end, the light guide 120 can be connected to an appropriate evaluation device which can read out the corresponding identity and application data saved in the transponder 130 and decipher and evaluate it. Preferably, the data in the transponder 130 can be encrypted by the above mentioned encryption unit when saved to protect it from tampering or forging. The data in the transponder 130 typically cannot be deleted, overwritten, or modified. In this way, it can be ensured that the data saved in the transponder 130 is essentially reproduced without forging for all light guide 120 applications. As a result, in the case of damage to the light guide 120, it is possible to trace in what way incorrect operation of the laser device 110 or non-conformance to the boundary conditions for operation of the light guide 120 are the cause of the damage. In this way, an assessment of whether a quality defect or non-conformance to the boundary conditions for the application of the light guide 120 is involved can be significantly simplified and a clear safety and reliability advantage can be established for the manufacturer of therapeutic fibers, especially expendable therapeutic fibers.

This invention is not restricted to the quoted preferred embodiments, but rather also extends to the combination of all preferred embodiments. Furthermore, this invention is not restricted to the field of medical applications, but rather can be used equivalently in the fields of material processing and material analysis. While the invention has been described with respect to the physical embodiments constructed in accordance therewith, it will be apparent to those skilled in the art that various modifications, variations, and improvements of the invention can be made in light of the above teachings and within in the purview of the appended claims without departing from the spirit and intended scope of the invention. In addition, those areas in which it is believed that those of ordinary skill in the art are familiar have not been described herein in order not to unnecessarily obscure the invention described herein. Accordingly, it is to be understood that the invention is not to be limited by the specific exemplary embodiments described herein, but only by the scope of the appended claims.

What is claimed is:

1. A laser system, comprising:
a laser device for the generation of laser radiation;
a light guide for guiding the generated laser radiation;
a data medium for identity data connected to the light guide; and
a readout device for reading out the identity data.

2. The laser system of claim 1, wherein the data medium is permanently connected to the light guide.

3. The laser system of claim 1, wherein the data medium is a transponder.

4. The laser system of claim 1, wherein the identity data contains information about at least one of a manufacturer of the light guide, an end date of use of the light guide, a transmission of the light guide, a type designation of the light guide, a maximum transmission power of the light guide, a fiber diameter of the light guide.

5. The laser system of claim 1, wherein application data is saved in the data medium regarding a specific application of the light guide in conjunction with the laser device.

6. The laser system of claim 5, wherein the application data contains information about at least one of a laser energy passed to the light guide, a number of treatments with the light guide, a date of the treatment with the light guide, or an identification data of the laser device, and wherein the application data is saved in the data medium using the memory device.

7. The laser system of claim 5, wherein the application data saved in the data medium cannot be deleted, overwritten, or modified.

8. The laser system of claim 1, wherein the identity data and the application data are saved encrypted in the data medium.

9. The laser system of claim 1, wherein the light guide is mounted in a releasable manner on the laser device using a mounting device.

10. The laser system of claim 9, wherein the data medium is essentially mounted inseparably in the part of the mounting device fitted to the light guide by at least one of the method of encapsulation, welding, and gluing.

11. The laser system of claim 9, wherein the mounting device is one of a plug, screw, and bayonet connection.

12. The laser system of claim 1, wherein the laser system is a medical laser system.

13. A light guide system, comprising:
a light guide for guiding laser radiation, wherein the light guide can be releasably coupled to a laser device using a mounting device; and
a data medium for identity data connected to the light guide.

14. The light guide system of claim 13, wherein the data medium is permanently connected to the light guide.

15. The light guide system of claim 13, wherein the identity data contains information about at least one of a manufacturer of the light guide, an end date for usage of the light guide, a transmission of the light guide, a type designation of the light guide, a maximum transmission power of the light guide, or a fiber diameter of the light guide.

16. The light guide system of claim 13, wherein the data medium is readable and writable in order to save application data about a specific application of the light guide in conjunction with a laser device.

17. The light guide system of claim 16, wherein the application data contains information about at least one of a laser energy passed to the light guide, a number of treatments with the light guide, a date for the treatment with the light guide, or an identification data of the laser device, and wherein application data already saved in the data medium cannot be deleted, overwritten, or modified.

18. The light guide system of claim 16, wherein the identity data and the application data are saved encrypted in the data medium.

19. The light guide system of claim 13, wherein the light guide with the mounting device is essentially connected inseparably and the transponder is welded to, glued to, or encapsulated in the mounting device.

20. The light guide system of claim 13, wherein the light guide is an expendable light guide.
